(19) United States
(12) Patent Application Publication
(10) Pub. No.: US 2005/0119687 A1
(43) Pub. Date: Jun. 2, 2005

(54) Methods of, and materials for, treating vascular defects with magnetically controllable hydrogels

(76) Inventors: Ralph G. Dacey JR., St. Louis, MO (US); Bevil J. Hogg, St. Louis, MO (US); Francis M. Creighton IV, St. Louis, MO (US); Peter R. Werp, St. Louis, MO (US); Rogers C. Ritter, Charlottesville, VA (US)

Correspondence Address: HARNESS, DICKEY, & PIERCE, P.L.C, 7700 BONHOMME, STE 400, ST. LOUIS, MO, 63105, US

(21) Appl. No.: 10/936,207

(22) Filed: Sep. 8, 2004

Related U.S. Application Data
(60) Provisional application No. 60/501,175, filed on Sep. 8, 2003.

(51) Int. Cl. A61M 29/00

(52) U.S. Cl. 606/200

(57) ABSTRACT

Embolic devices and materials include an expansible polymer, and magnetically responsive material that allow the embolic devices and materials to be guided into, and held within, vascular defects, while the expansible polymer expands. In some embodiments, the expansion of the expansible polymer reduces the density of the magnetic material, so that subsequent magnetic surgery and magnetic imaging procedures can still employed.

### CROSS-REFERENCE TO RELATED APPLICATIONS

This application claims priority of U.S. Provisional Patent Application Ser. No. 60/501,175, filed Sep. 8, 2003, the disclosure of which is incorporated by reference.

### BACKGROUND OF THE INVENTION

This invention relates to methods of and apparatus for treating vascular defects, such as aneurysms and atriovenous malformations, and in particular to methods of, and materials for, treating such defects with magnetically manipulated hydrogels.

### BACKGROUND OF THE INVENTION

There are many types of vascular defects that can be treated by blocking the defect. One example of such a defect is an aneurysm, which is a permanent, abnormal blood-filled dilatation or ballooning of a blood vessel, which may be congenital or the result of disease. Aneurysms typically have thin walls vulnerable to rupture. If an aneurysm ruptures, the resulting hemorrhage can put injurious pressure on surrounding tissue, impair downstream blood flow, and even cause death. Another example of a vascular defect is an atriovenous malformation—a typically congenital shunt formed between an artery and a vein that often carries a substantial blood flow. One of the principal complications in treating these and other vascular defects is the blood flow in the adjacent vessels which impairs treatment, but should be maintained for the health of the patient.

Current treatments for aneurysms include embolizing the aneurysm to remove the dilatation or balloon from the wall of the vessel. In the most mature technique, the surgeon accesses the region of the aneurysm under direct visualization and places one or more aneurysm clips on the opening or “neck” of the aneurysm. While this conventional surgical technique has a high rate of success, it is highly invasive and for that reason it is undesirable. More recently, less invasive techniques have been developed for the treatment of aneurysms. One such technique involves the introduction of small wire coils into the aneurysm. A catheter is navigated to the site of the aneurysm, and the coils are delivered through the lumen of the catheter into the aneurysm. The coils reduce the blood flow through the aneurysm, which results in clotting within the aneurysm. An example of a coil for such procedures is disclosed in U.S. Pat. No. 6,605,101, incorporated herein by reference. This coiling procedure can be time consuming both in navigating the catheter through the vasculature to the site of the aneurysm, and in introducing the coils into the aneurysm. In some cases, the shape of the aneurysm allows the coils to escape from the aneurysm, requiring the coil to be retrieved and replaced.

Another less invasive technique for treating vascular defects is the delivery of embolic materials to the site of the vascular defect to occlude the defect. In the case of an aneurysm a balloon is inflated over the neck of the aneurysm and a liquid embolic agent is introduced into the aneurysm. Attempts have been made to deliver embolic agents directly into the dilation or balloon of the aneurysm. Embolic agents have also been used to occlude atriovenous malformations, but it can be difficult to accurately deliver the embolic agents.

More recently, hydrogels and elements coated with hydrogels have been used in the embolization of vascular defects. Examples of such procedures are disclosed in U.S. Pat. No. 6,602,261, entitled Filamentous Embolic Device with Expansile Elements, incorporated herein by reference.

### SUMMARY OF THE INVENTION

The invention provides devices, materials and methods for treating vascular defects by occluding them. Broadly, the invention relates to embolizing devices and materials for occluding vascular defects, and to methods for magnetically delivering and using embolizing devices and materials for occluding vascular defects.

### BRIEF DESCRIPTION OF THE DRAWINGS

FIG. 1 is a side elevation view of a first embodiment of an embolization device constructed according to the principles of this invention, prior to expansion of the expansile element(s);

FIG. 2A is an enlarged partial longitudinal cross-sectional view of a first alternate construction of the embolization device of the first embodiment, prior to expansion of the expansile elements;

FIG. 2B is an enlarged transverse cross-sectional view of the embolization device shown in FIG. 2A, taken along the plane of line 2B-2B in FIG. 2A;

FIG. 3A is an enlarged partial longitudinal cross-sectional view of the embolization device shown in FIG. 2, after expansion of the expansile elements;

FIG. 3B is an enlarged transverse cross-sectional view of the of the embolization device shown in FIG. 3A, taken along the plane of line 3B-3B in FIG. 3A;

FIG. 4A is an enlarged partial longitudinal cross-sectional view of a second alternate construction of the embolization device of the first embodiment, prior to expansion of the expansile elements;

FIG. 4B is an enlarged transverse cross-sectional view of the embolization device shown in FIG. 4A, taken along the plane of line 4B-4B in FIG. 4A;

FIG. 5A is an enlarged partial longitudinal cross-sectional view of the embolization device shown in FIG. 4, after expansion of the expansile elements;

FIG. 5B is an enlarged transverse cross-sectional view of the embolization device shown in FIG. 5A, taken along the plane of line 5B-5B in FIG. 5A;

FIG. 6A is an enlarged partial longitudinal cross-sectional view of a third alternate construction of the embolization device of the first embodiment, prior to expansion of the expansile elements;

FIG. 6B is an enlarged transverse cross-sectional view of the embolization device of FIG. 6A, taken along the plane of line 6B-6B in FIG. 6A;

FIG. 7A is an enlarged partial longitudinal cross-sectional view of the embolization device of FIG. 6, after expansion of the expansile elements;

FIG. 7B is an enlarged transverse cross-sectional view of the embolization device of the first embodiment, taken along the plane of line 7B-7B in FIG. 7A;

FIG. 8A is an enlarged partial longitudinal cross-sectional view of a fourth alternate construction of the embolization device of the first embodiment, prior to expansion of the expansile elements;

FIG. 8B is an enlarged transverse cross-sectional view of the embolization device of FIG. 8A, taken along the plane of line 8B-8B in FIG. 8A;

FIG. 9A is an enlarged partial longitudinal cross-sectional view of the embolization device of FIG. 8, after expansion of the expansile elements;

FIG. 9B is an enlarged transverse cross-sectional view of the embolization device of FIG. 9A, taken along the plane of line 9B-9B in FIG. 9A;

FIG. 10A is a perspective view of a second embodiment of an embolization device constructed according to the principles of this invention, prior to expansion of the expansile element, prior to expansion;

FIG. 10B is a perspective of the embolization device of FIG. 10A, after expansion;

FIG. 11A is a schematic view of the embolization device of FIG. 10 being deployed from a microcatheter into a vascular defect;

FIG. 11B is a schematic view of the embolization device of FIG. 10, after deployment into the vascular defect;

FIG. 11C is a schematic view of the embolization device after it is aligned in the vascular defect by the application of a magnetic field;

FIG. 11D is a schematic view of the embolization device after it expands in the correct orientation to occlude the vascular defect.

FIG. 12A is a cross-sectional view of a particle comprising the embolic material in accordance with the principles of a second embodiment of this invention, prior to expansion of the expansile coating;

FIG. 13A is schematic diagram of the delivery of an embolization device into a vascular defect in accordance with one embodiment of a method of this invention;

FIG. 13B is a schematic diagram of the expansion of the embolic device in the vascular defect as it is being held in place;

FIG. 14A is schematic diagram of the delivery of an alternative embolization device into a vascular defect in accordance with one embodiment of a method of this invention;

FIG. 14B is a schematic diagram of the expansion of the embolic device in the vascular defect as it is being held in place;

FIG. 15A is schematic diagram of the delivery of an alternative embolization device into a vascular defect in accordance with one embodiment of a method of this invention;

FIG. 15B is a schematic diagram of the expansion of the embolic device in the vascular defect as it is being held in place; and

FIG. 16 is a schematic diagram the delivery of embolic material of the third embodiment in accordance with one embodiment of the method of this invention.

Corresponding reference numerals indicate corresponding parts throughout the several views of the drawings.

### DETAILED DESCRIPTION OF THE INVENTION

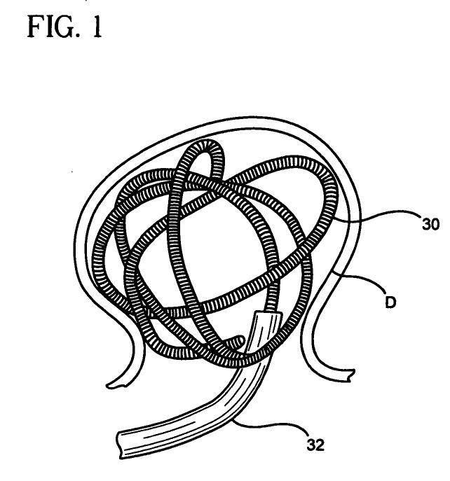

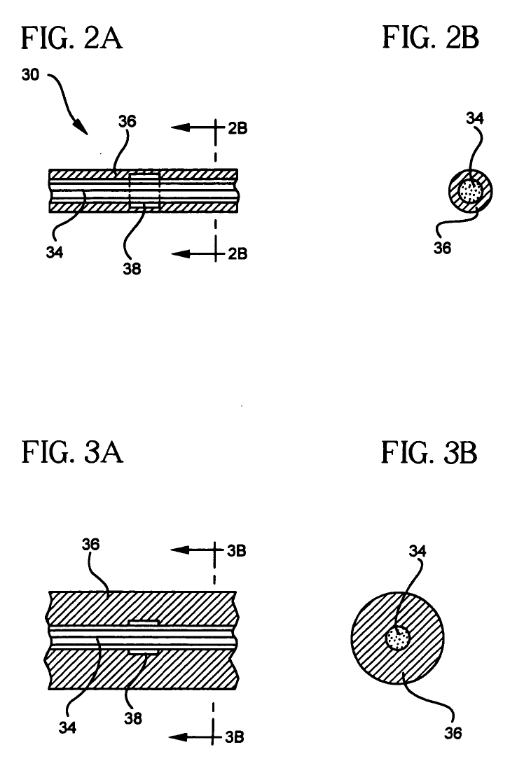

A first embodiment of a vascular embolization device constructed according to the principles of the present invention, indicated generally as 30 in FIG. 1, is shown as it might be introduced into a vascular defect D, such as an aneurysm, from a microcatheter 32. Various constructions of the embolization devices of this first embodiment are shown in FIGS. 2-9, and generally comprise an elongate, flexible, carrier 34. At least one expansile element 36 is fixed to the carrier 34. The expansile element 36 is preferably formed from an expansile polymer that expands in response to introduction of the device into the body. A magnetically responsive material associated with the carrier 34 or the expansile element 36. There is preferably sufficient magnetic material to create a pulling force of 0.5 g/cc on the device 20 in an applied magnetic gradient of at least 0.5 T/m.

The carrier 34 can be a single polymer filament or metal wire, or a braid of such filaments or wires, or a coil (e.g., helical coil) of such filaments or wire. The carrier 34 may be made of a magnetically responsive material, i.e. either a permanent magnetic material or a permeable magnetic material which is attracted in a magnetic gradient, aligns relative to a magnetic field, or both. Rather than being made in whole or in part of a magnetically responsive material, the carrier 34 can carry one or more elements of magnetically responsive material. The carrier may be made of a shape memory material so that it tends to return to a shape selected to enhance filling of the vascular defect.

The expansile element(s) 36 may be a single element disposed on the carrier 34, it may be a plurality of elements 36 disposed on the carrier in spaced relationship (for example a plurality of expansile filaments), or it may constitute a layer or coating on all or substantially all of the carrier 34. The expansile element 36 is made of expansile polymer can be hydrophilic, macroporous, polymeric, hydrogel foam material, or a porous, environmentally-sensitive, expansile hydrogel that expands, after a predetermined time delay, in response to a change in an environmental parameter, such as pH or temperature, corresponding to an environmental change resulting from instruction into the vascular defect in the subject's body. Examples of suitable materials are know in the art, and are disclosed for example in U.S. Pat. No. 6,602,261, Filamentous embolic device with expansile elements, incorporated herein by reference, as well as in U.S. Pat. No. 6,607,538, Mechanism for the deployment of endovascular implants; U.S. Pat. No. 6,605,101, Microcoil vaso-occlusive device with multi-axis secondary configuration; U.S. Pat. No. 6,602,261, Filamentous embolic device with expansile elements; U.S. Pat. No. 6,537,569, Radiation cross-linked hydrogels; U.S. Pat. No. 6,511,492, Embolectomy catheters and methods for treating stroke and other small vessel thromboembolic disorders; U.S. Pat. No. 6,500,190, Vascular embolization with an expansible implant; U.S. Pat. No. 6,375,669, Apparatus and method for vascular embolization; U.S. Pat. No. 6,299,619, Methods for embolizing a target vascular site; U.S. Pat. No. 6,238,403, Filamentous embolic device with expansible elements; U.S. Pat. No. 6,165,193, Vascular embolization with an expansible implant; U.S. Pat. No. 6,015,424, Apparatus and method for vascular embolization, the disclosures of which are incorporated herein by reference.

The expansile polymer preferably responds to a change in environment accompanying the introduction of the device 30 into the body. For example, the expansile polymer can respond to a change in pH accompanying exposure to the blood after introduction into the body. Alternatively, the expansile can respond to a change in temperatures that accompanies the introduction of the device 30 into body, which is typically at 98.6 F (37 C). Alternatively the expansile polymer can respond to some other environmental parameter.

The expansile element 36 could be a single element covering all or substantially all of the surface of the carrier. Alternatively, a plurality of discrete expansile elements 36 could be provided. The expansile element or elements 36 can be shaped to expand to a shape that substantially conforms to the size and shape of the vascular defect.

The magnetically responsive material could be incorporated into the expansile polymer, for example being disbursed as microparticles or nanoparticles in the expansile polymer. The particles are preferably generally spherical, and thus the size can be characterized by diameter, which is preferably less than about 50 nm. However, the particles may be elongate, in which case the long dimension is preferably less than about 50 nm.

When the magnetic material is incorporated into the expansile polymer, the density of the magnetic material will change with the expansion of the expansile polymer, decreasing as the volume of the expansile polymer increases. The initial density of the magnetically responsive material is preferably sufficiently high that an applied magnetic gradient can hold the device 30 against the hemodynamic forces of blood flowing through blood vessels adjacent the vascular defect. An external source magnetic can conveniently apply a magnetic gradient on the order of 0.5 T/m, and the density of the magnetically responsive material is preferably sufficient to be held against the hemodynamic forces in the vasculature by such a gradient.

Upon expansion of the expansile polymer, the density of the magnetically responsive material decreases, preferably to below a level that would interfere with subsequent medical procedures, such as magnetic resonance imaging and magnetic surgery procedures. Prior to expansion, it is desirable that the density of the magnetic material is greater than about 1 percent by volume. After expansion, the density of magnetic material is preferably below about 3 percent by volume, and more preferably below about 1 percent by volume. After the embolic device 30 is deployed in a vascular defect, it is desirable that a magnetic gradient from an MRI does not exert a pulling force that is harmful to the subject. Prior to expansion, it is desirable that the pulling force on the embolic device 30 in a magnetic gradient of 0.5 T/m be at least 0.5 gm/cc, so that the magnetic gradient can adequately hold and control the device. After expansion it is desirable that the pulling force on the device be less than 5 gm/cc in a magnetic gradient of 1 T/m, and preferably less than about 3 gm/cc in a 1 T/m gradient. The pulling force is more preferably less than about 5 gm/cc in a 10 T/m gradient, and still more preferably less than about 3 gm/cc in a 10 T/m gradient.

Instead of, or in addition to, being engagable by a magnetic gradient, the embolization device 30 can be magnetically orientable. The magnetically responsive material can include a permanent magnetic material whose magnetization direction is oriented in a convenient direction for orienting the device or portions of the device with a magnetic field. Alternatively the magnetically responsive material can include a permeable magnetic material, and be shaped so that the material is orientable in a magnetic field. The embolization device 30 can either have portions made of magnetic material, or can include magnetic elements attached to the carrier 34 or embedded in the expansile polymer, whose magnetization directions are substantially aligned prior to expansion of the expansile element 36 so that the device 30 can be aligned with an applied magnetic field. After expansion, this alignment may or may not remain. Alternatively, the magnetically responsive material can be a permeable magnetic material shaped to have a preferred directions of magnetization which are substantially aligned prior to expansion of the element so that the device can be aligned with an applied magnetic field. After expansion, this alignment may or may not remain. The magnetically responsive material is preferably such that in a magnetic field of at least 0.05T, an aligning torque of at least 1 g-cm/cc is applied to the device.

In a first construction of the embolization device of the first embodiment shown in FIGS. 2 and 3, the carrier 34 is a flexile, fibrous filament. The expansile element 36 is a coating of an expansile polymer on all or substantially all of the carrier 34. The magnetic material is in the form of one or more magnet elements 38 on the carrier 34. the magnet element(s) 38 can be a ring (or other shape) of a permanent magnetic material, such as a Nd—Fe—B alloy, or it can a ring (or other shape) of a permeable material, such as hiperco. The device 30 of FIG. 2 can be introduced into a vascular defect, such as an aneurysm, through a microcatheter 32, and held in place with the application of a magnetic gradient. Once in the vascular defect, the expansile element 36 expands, as shown in FIG. 3, filling and occluding the vascular defect. This allows clots to form in the defect, eventually completely filling and blocking the defect. Eventually, epithelial cells will grow over the occlusion, completely isolated the occlusion from the vascular system.

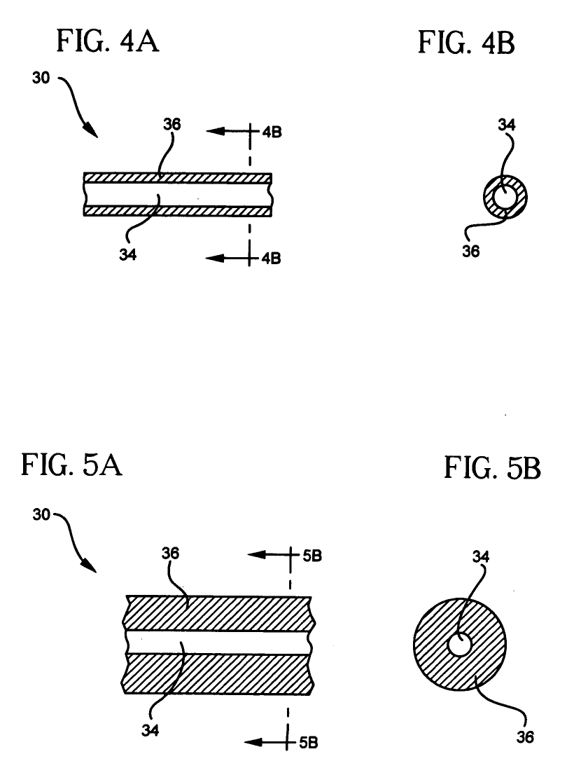

In a second construction of the embolization device of the first embodiment shown in FIGS. 4 and 5, the carrier 34 is a thin flexile element made of a magnetically responsive material, either permanently magnetic or permeable. The expansile element 36 is a coating of an expansile polymer on all or substantially all of the carrier 34. As noted above, the magnetic material is incorporated into the carrier 34. The device 30 of FIG. 4 can be introduced into a vascular defect, such as an aneurysm, through a microcatheter 32, and held in place with the application of a magnetic gradient. Once in the vascular defect, the expansile element 36 expands, as shown in FIG. 5, filling and occluding the vascular defect. This allows clots to form in the defect, eventually completely filling and blocking the defect. Eventually, epithelial cells will grow over the occlusion, completely isolated the occlusion from the vascular system.

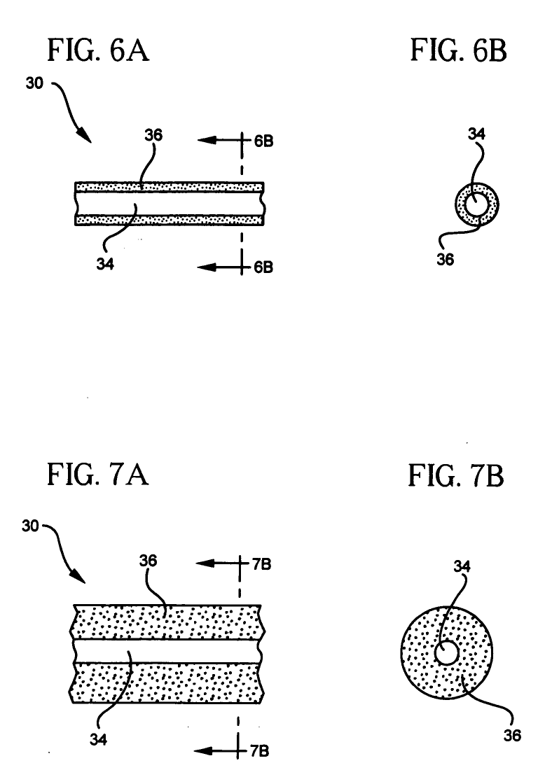

In a third construction of the embolization device of the first embodiment shown in FIGS. 6 and 7, the carrier 34 is a thin flexible element. The expansile element 36 is a coating of an expansile polymer on all or substantially all of the carrier 34. The magnetic material is incorporated into the expansile element 36. The device 30 of FIG. 6 can be introduced into a vascular defect, such as an aneurysm, through a microcatheter 32, and held in place with the application of a magnetic gradient. Once in the vascular defect, the expansile element 36 expands, as shown in FIG. 7, filling and occluding the vascular defect. As the expansile element expands, the density of magnetically responsive material decreases, and the hold of the magnetic gradient on the device 30 decreases. However, as the expansile element 36 expands, it helps retain the device in the vascular defect. This allows clots to form in the defect, eventually completely filling and blocking the defect. Eventually, epithelial cells will grow over the occlusion, completely isolated the occlusion from the vascular system. Because the final density of magnetically responsive material is relatively low, the subject is not necessarily excluded from future magnetic surgical procedures or magnetic resonance imaging procedures.

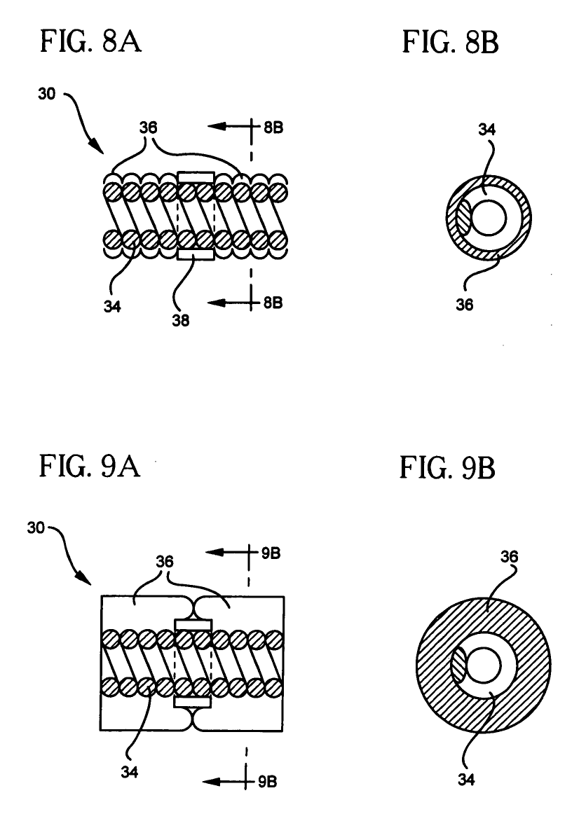

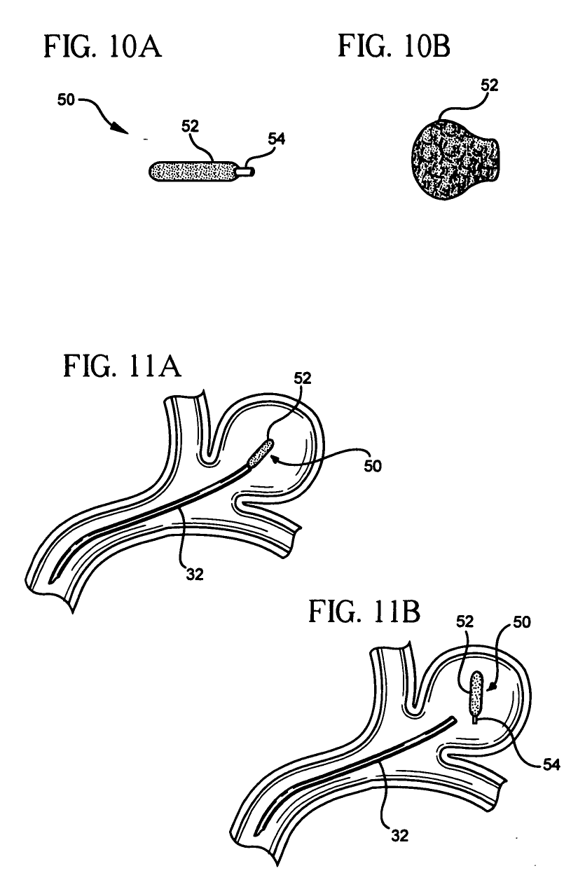

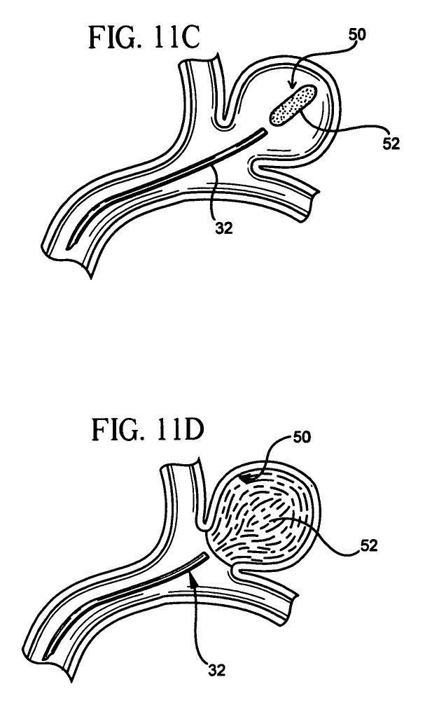

In a fourth construction of the embolization device of the first embodiment shown in FIGS. 8 and 9, the carrier 34 is a flexile, helical element, which may be made from a polymeric material, a non-magnetic metallic material, or a magnetic metallic material. The expansile element 36 is a plurality of elements spaced along the length of the device 30. The magnetic material can be in the form of one or more magnet elements 38 on the carrier 34. The magnet element(s) 38 can be a ring (or other shape) of a permanent magnetic material, such as a Nd—Fe—B alloy, or it can a ring (or other shape) of a permeable material, such as hiperco. In stead of or in addition to the elements 38, the carrier can be made of a magnetically responsive material, or magnetically responsive material can be incorporated into the expansile elements 36. The device 30 of FIG. 8 can be introduced into a vascular defect, such as an aneurysm, through a microcatheter 32, and held in place with the application of a magnetic gradient. Once in the vascular defect, the expansile element 36 expands, as shown in FIG. 9, filling and occluding the vascular defect. This allows clots to form in the defect, eventually completely filling and blocking the defect. Eventually, epithelial cells will grow over the occlusion, completely isolated the occlusion from the vascular system.

Some portion of the device, e.g., the carrier 22, the embolizing elements 24, or the magnetically responsive material is preferably radiopaque, so that the device can be viewed on x-rays. Alternatively, or in addition, portions of the device can be made visible to other imaging methods, such as mr imaging.

In accordance with a second embodiment of this invention, an expandable embolization device can expand from a initial size and shape, to a size and shape designed to fit or substantially fit the vascular defect. Such a device might have an initial configuration in which it is in the form of a model of the vascular defect, and the device is then compressed from this initial configuration into a compressed configuration, but is expansible from the compressed configuration into an expanded configuration substantially conforming to the shape and size of the vascular defects. The device preferably includes at least one magnetically responsive element therein capable of aligning the device in an applied magnetic field of at least 0.05T.

In a second embodiment of the invention, an embolic device comprises an embolic material comprises an expansible hydrogel body with a magnetically responsive material associated therewith. The magnetically responsive material preferably creating a pulling force of at least about 0.5 g/cc in a magnetic gradient of 0.5T/m, prior to expansion of the hydrogel. The magnetically responsive material comprises an element of a magnetically responsive material embedded in the hydrogel body. Alternatively, the magnetically responsive material can comprises a magnetically responsive coil. Alternatively, the magnetically responsive material comprise particles of magnetic material embedded in hydrogel In whatever form, the magnetic density of the magnetically responsive material before expansion of the hydrogel is greater than about 0.75 percent by volume and wherein the density of the magnetic material after expansion of the hydrogel is less than about 0.5 percent by volume, and the pulling force on the material in a gradient of 0.5 T/m is less than about 0.25 g/cc after expansion of the hydrogel. In addition the embolic device is preferably alignable with an applied magnetic field of at least 0.05T to orient the body in a selected orientation.

A device constructed in accordance with the principles of this invention is indicated generally as 50 in FIGS. 10 and 11. The embolization device 50 comprises a body 52 of an expansile polymer, which may be the hydrogel material identified above. The embolization device 50 further comprises magnetically responsive material. The magnetically responsive material is preferably in the form of an elongate element 54 of a permanent magnet material or a permeable magnetic material. The magnetic material could also be in the form of microparticles or nanoparticles disposed in the body 52 of the expansile polymer.

The element 54 is preferably at least responsive to a magnetic gradient allowing the device 52 to be moved by and held by a magnetic gradient applied to the device. However, the device is preferably also responsive to an applied magnetic field, tending the align relative to an applied magnetic field. Thus, as shown in FIG. 11A, a the device 50 can be delivered from a microcatheter 32 into a vascular defect D. As shown in FIG. 11B, the device assumes a random orientation in the vascular defect D. However, as shown in FIG. 11C, upon the application of a magnetic field of appropriate direction, the device 50 tends to align relative to the applied magnetic field, in a desired direction. The device 50 can be held in the desired position and orientation as the expansile body 52 expands to fill in the defect D. The element 54 makes it easier to align the device 50, but the same effect could be achieved with a device in which the magnetic material is disbursed in the body 52, with the advantage that the magnetic responsive can be made to diminish as the expansile material expands.

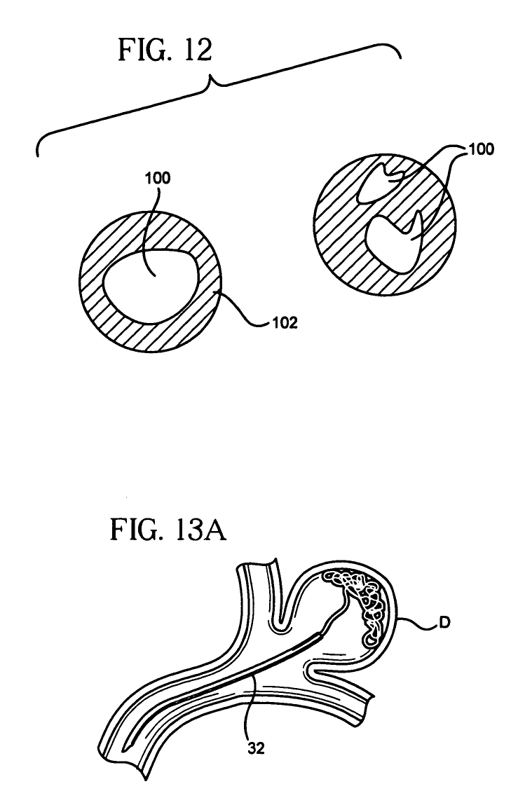

An embolic material in accordance with a third embodiment of this invention generally comprises magnetically responsive particles coated with an expansile material. As shown in FIG. 12, the embolic material preferably comprises a plurality of such particles, which may be included in fluid carrier, as is known in the art. As shown in FIG. 12, a core consisting of one or more particles 100 of magnetically responsive material are at least partially coated with a layer 102 of expansile material, such as the hydrogel discussed above. The cores 100 are preferably made of a magnetically responsive material, such as magnetite (Fe₃O₄). The cores 100 could also be hematite (Fe₂O₃), cobalt, iron, mixtures or alloys thereof, or other magnetic particles which could be made biologically compatible, for example with coatings. The magnetic particles preferably comprise magnetic bodies, preferably made of a permeable magnetic material, such as the iron oxides magnetite (Fe₃O₄) or maghemite (Fe₂O₃), or ferrites of the general form MO—Fe₂O₃, where M stands for Fe, Ni, Mn, Co, or Mg. Most superparamagnetic, ferromagnetic, and ferrimagnetic metal alloys and garnets may also be used as magnetic bodies. Examples are Pt/Fe (ferromagnetic alloy) and R₃Fe₅Ol₂ (where R=atomic number 39, 62-71, ferromagnetic garnets). It would be desirable if the particles were radiopaque, so that the delivery of the particles could be monitored by x-ray or fluoroscope. Thus the particles could include, for example, barium in the form of a barium iron oxide, e.g., BaO.Fe₂O₃, gadolinium, or europium or other suitable radiopaque material. Of course all of the cores 100 do not have to have the same composition, and portions of the particles could have cores of different materials to provide particular properties to the embolic material.

The coating can serve at least two functions: First, it can help keep the particles 100 separated so that they do not undesirably agglomerate when a magnetic field is applied to them. Second, the coating expands upon delivery to the vascular defect, helping to occlude the defect.

In accordance with a third embodiment of this invention, an embolic material is provided for occluding vascular defects, the embolic material comprising a plurality of magnetically responsive particles each coated with a polymer that expands upon deployment in the vascular defect, for example when contacted with blood. The particles are preferably generally spherical with a diameter of less than about 50 nm. However, the particles could be elongate, in which case the longest dimension of the particles is preferably less than about 50 nm, and more preferably between about 5 nm and about 50 nm.

The polymer coating is preferably a hydrogel material, discussed above. The coating may be continuous, i.e., uninterrupted, or it may have areas of discontinuity. The coating serves as a buffer to maintain a desirable inter-particle spacing. This inter-particle spacing actually increases as the polymer expands. For example, the coating on the particles may have a thickness of between about 2.5 nm and about 20 nm prior to introduction into the vascular defect, and a thickness of between about 5 nm and about 50 nm after introduction into the vascular defect. As a result of the expansion of the polymer, the magnetic density of the material decreases after introduction into the vascular defect. For example, the material can have a magnetic density of between about 0.5 g/cc and about 2 g/cc before the polymer expands, and a magnetic density of between about 0.75 g/cc and about 3 g/cc after the polymer expands, and most preferably a magnetic density of less than about 0.5 g/cc after the hydrogel expands. The force on the embolic material prior to expansion of the polymer in a magnetic gradient of 0.5 T is at least 0.5 g/cc, and the magnetic force on the embolic material after expansion of the hydrogel in a magnetic gradient of 1 T/m is preferably less than 5 g/cc, and more preferably the magnetic force on the embolic material after expansion of the hydrogel in a magnetic gradient of 10 T/m is less than 5 g/cc.

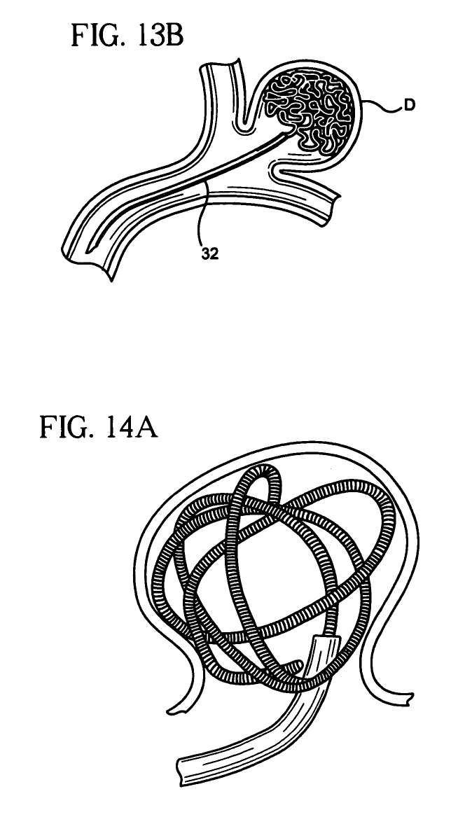

FIG. 13 generally illustrates the magnetically assisted embolization in accordance with the methods of this invention. A microcatheter 32 is navigated (either conventionally or magnetically) to the site of the vascular defect, and one or more of the devices 30 of the first embodiment is ejected into the defect. As shown in FIGS. 13A and 13B, a magnetic gradient applied to the devices 30 helps to guide and retain the devices in the defect.

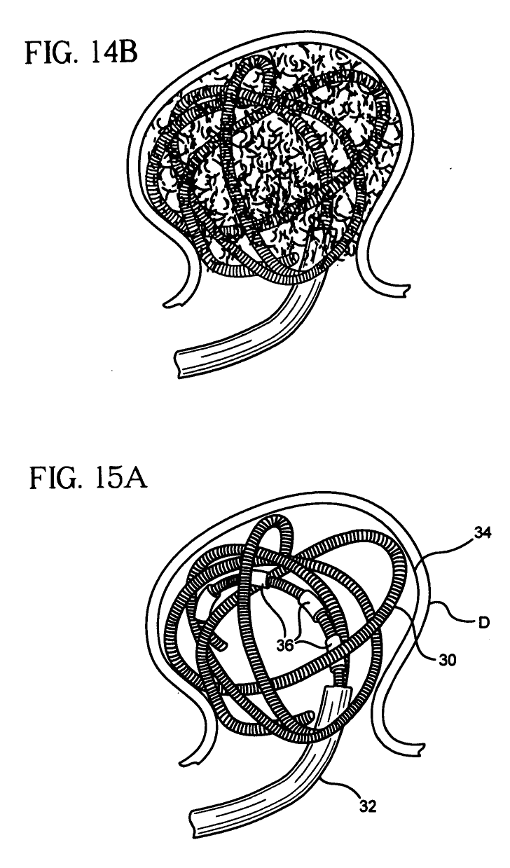

FIG. 14 illustrates a method of treating a vascular defect in using an embolizing device 30 in accordance with the first embodiment of this invention. As shown in FIG. 14A. the device 30 is introduced through a microcatheter 32 into the vascular defect. A magnetic gradient is applied to hold the device in the vascular defect while, as shown in FIG. 14B, the expansible element 36 on the exterior of the carrier 34 expands to fill the vascular defect.

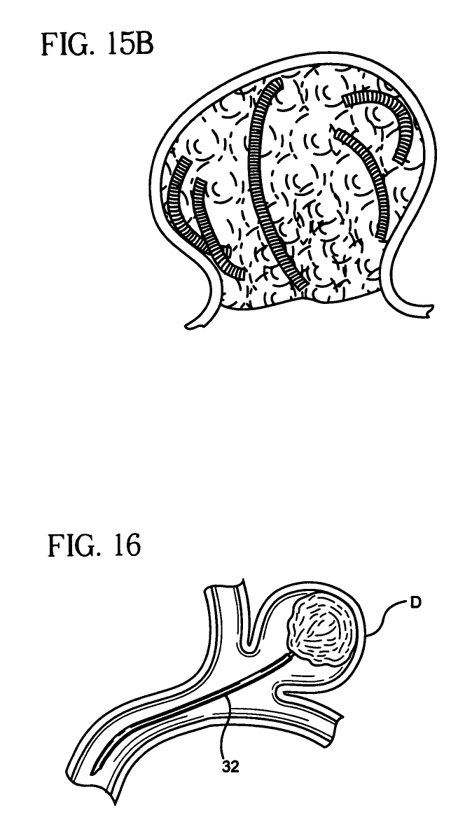

FIG. 15 illustrates a method of treating a vascular defect using an embolizing device 30 in accordance with the first embodiment of this invention. As shown in FIG. 15A. the device 30 is introduced through a microcatheter 32 into the vascular defect. A magnetic gradient is applied to hold the device in the vascular defect while, as shown in FIG. 15B, the discrete expansible elements 36 on the exterior of the carrier 34 expands to fill the vascular defect.

A method of treating a vascular defect using a device 50 in accordance with the second embodiment of this invention is illustrated in FIG. 11. In accordance with the method, and as shown in FIG. 11A, the device 50 comprising a expansile polymer body 52 and associated magnetically responsive element 54 is introduced into the vascular defect. As shown in FIGS. 11B and 11C, a magnetic field is applied to the device the embolizing element in a direction appropriate to orient the embolizing element in a selected direction in the defect while the expansile polymer expands. As shown in FIG. 11D, the embolizing element can be constructed so that in its expanded condition, it assumes a size and/or shape to occlude the vascular defect. The embolizing element can be aligned in the appropriate orientation so that it properly fits within and occludes the defect by the application of a magnetic field in a direction selected to cause the magnetically responsive material to orient in the desired direction, thereby aligning the associated embolizing element.

Broadly, various embodiments of the methods of treating a vascular defect in accordance with a third embodiment of this invention comprises introducing a embolizing element comprising a expansile polymer and associated magnetically responsive material into the vascular defect. A magnetic field and gradient of selected directions is applied to the embolizing element in the vascular defect to orient the embolizing element in the defect and to hold the embolizing element in the defect while the expansile polymer expands. By controlling the direction of the applied field, the orientation of the embolizing element or elements can be controlled. By controlling the direction of the applied gradient, the direction of the pulling force tending to hold the embolizing element or elements can be controlled. More specifically, a microcatheter is navigated intravascularly so that its distal end is introduced into a target vascular defect. A vascular embolization device or material comprising an expansible embolizing element through the microcatheter so that it emerges from the distal end of the microcatheter into the target vascular site; and applying a magnetic gradient to drawing the device or material into and hold the device or material in hold the device in the vascular site while permitting the embolizing element to expand in situ substantially to fill remaining volume of the target vascular site while retaining the embolizing element on the carrier.

1. A vascular embolization device, comprising:
an elongate, flexible, filamentous carrier;
at least one embolizing element fixed to the carrier, the embolizing element being formed from an expansile polymer that expands in response to a change exposure to blood; and
magnetically responsive material associated with the carrier or the at least one element, sufficient to create a pulling force of 0.5 g/cc on the device in a magnetic gradient of at least 0.5 T/m.

2. A vascular embolization device, comprising:
an elongate, flexible, filamentous carrier;
at least one embolizing element fixed to the carrier, the embolizing element being formed from an expansile polymer that expands in response to a change exposure to blood; and
magnetically responsive material associated with the carrier or the at least one element, sufficient to hold the device against the hydrodynamic force of blood flowing past the device, in a magnetic gradient of at least 0.5 T/m.

3. The device according to claim 2 wherein the magnetically responsive material comprises at least one magnetically responsive element attached to the carrier.

4. The device according to claim 3 wherein the magnetically responsive material comprises particles of magnetically responsive material in the embolizing element.

5. The device according to claim 4 wherein the density of magnetically responsive material is at least about one percent by volume prior to expansion of the embolizing element.

6. The device according to claim 5 wherein the density of the magnetically responsive material is no more than about three percent after expansion of the embolizing material.

7. The device according to claim 4 wherein the density of the magnetically responsive material is no more than about three percent after expansion of the embolizing material.

8. The device according to claim 4 wherein the density of magnetically responsive particles prior to expansion of the embolizing element is sufficient to create a force of at least 0.5 g/cc on the device in a magnetic gradient of 0.5 T/m.

9. The device according to claim 8 wherein the density of magnetically responsive particles after expansion of the embolizing element is such that it does not create a force greater than 5 g/cc in an magnetic gradient of 1 T/m.

10. The device according to claim 8 wherein the density of magnetically responsive particles after expansion of the embolizing element is such that it does not create a force greater than 5 g/cc in an magnetic gradient of 10 T/m.

11. The device according to claim 8 wherein the magnetically responsive material is a permanent magnetic material whose magnetization directions are substantially aligned prior to expansion of the element so that the device can be aligned with an applied magnetic field.

12. The device according to claim 11 wherein after expansion of the magnetic element, the magnetization directions of the particles are not substantially aligned.

13. The device according to claim 8 wherein the magnetically responsive material is a permeable magnetic material, and wherein the particles have preferred directions of magnetization which are substantially aligned prior to expansion of the element so that the device can be aligned with an applied magnetic field.

14. The device according to claim 13 wherein after expansion of the magnetic element, the preferred directions of magnetization of the particles are not substantially aligned.

15. The device according to claim 2 wherein the carrier is made from a magnetically responsive material.

16. The device according to claim 15 wherein the carrier is in the form of a helical coil.

17. The device according to claim 16 wherein the embolizing element substantially covers the surface of the carrier.

18. A vascular embolization device, comprising:
an elongate, flexible, filamentous carrier;
an embolizing element fixed to the carrier, the embolizing element being formed from an expansile polymer that expands in response to a change in an environmental parameter selected from the group consisting of temperature and pH after a predetermined time interval; and
a magnetically responsive element on the carrier.

19. The embolization device of claim 18 wherein the polymer is a porous hydrogel.

20. The embolization device of claim 18, wherein the embolizing element comprises a plurality of expansile polymer fibers attached to the carrier.

21. The embolization device of claim 18, wherein at least a portion of the embolizing element is radiopaque.

22. A vascular embolization device, comprising:
an elongate, flexible, filamentous carrier of predetermined length;
a plurality of expansile fibers fixed to the carrier at spaced intervals along the length of the carrier, the fibers being formed from an expansile polymer.

23. The embolization device of claim 22, wherein the polymer hydrogel is of a type that expands in response to a change in an environmental parameter selected from the group consisting of temperature and pH after a predetermined time interval.

24. A vascular embolization device, comprising:
a flexible, filamentous carrier having an exterior surface and a distal tip;
at least one expansile embolizing element non-releasably fixed coaxially to the exterior surface of the carrier at a location proximal from the distal tip, wherein the embolizing element is formed of an expansile, hydrophilic polymer that changes its physical character in response to a change in an environmental parameter selected from the group consisting of pH and temperature after a predetermined time interval;
and a magnetically responsive material associated with the carrier or the at least one expansile embolizing element, sufficient to create a force of at least 0.5 g/cc in an applied magnetic gradient of 0.5 T/m.

25. The device of claim 24, wherein the polymer is a porous hydrogel.

26. A vascular embolization device, comprising:
a flexible, filamentous carrier having an exterior surface and a distal tip;
at least one expansile embolizing element non-releasably fixed coaxially to the exterior surface of the carrier at a location proximal from the distal tip, wherein the embolizing element is formed of an expansile, hydrophilic polymer that changes its physical character in response to a change in an environmental parameter selected from the group consisting of pH and temperature after a predetermined time interval;
and a magnetically responsive material associated with the carrier or the at least one expansile embolizing element, sufficient to create an aligning torque of at least 1 g-cm/cc in an applied magnetic field of 0.05T.

27. The device of claim 26, wherein the polymer is a porous hydrogel.

28. The embolization device of claim 26, wherein the carrier comprises a material that is visible under X-rays.

29. The embolization device of claim 26, wherein the polymer is an environmentally-sensitive polymer that changes its physical character in response to a change in an environmental parameter selected from the group consisting of temperature and pH.

30. The embolization device of claim 26, wherein at least a portion of the embolizing element is radiopaque.

31. A method for treating a vascular defect, the method comprising the steps of:
introducing an embolizing element comprising a expansile polymer and associated magnetically responsive material into the vascular defect;
applying a magnetic gradient to the embolizing element to hold the embolizing element in the defect while the expansile polymer expands.

32. A method for treating a vascular defect, the method comprising the steps of:
introducing a embolizing element comprising a expansile polymer and associated magnetically responsive material into the vascular defect;
applying a magnetic field to the embolizing element to orient the embolizing element in the defect while the expansile polymer expands.

33. A method for treating a vascular defect, the method comprising the steps of:
introducing a embolizing element comprising a expansile polymer and associated magnetically responsive material into the vascular defect;
applying a magnetic field and magnetic gradient of selected directions to orient the embolizing element in the defect and to hold the embolizing element in the defect while the expansile polymer expands.

34. A vascular implant device for embolizing a vascular site, the device being formed from a hydrophobic hydrogel material and having an initial configuration for the embolization of a vascular aneurysm from which it is expansible primarily by hydrophobic action into an expanded configuration, and a magnetically responsive material associated with the device.

35. The vascular implant device of claim 34, wherein the implant device is compressible into its initial configuration from its expanded configuration.

36. The vascular implant device of claim 34, wherein the device is radiopaque.

37. The vascular implant device of claim 34, wherein the device is expansible into an expanded configuration that substantially conforms to the size and shape of the vascular site.

38. The vascular implant device of claim 34, wherein the device is formed as a unitary molded element.

39. A method for embolizing a target vascular site having a defined volume, comprising the steps of:
(a) passing a microcatheter intravascularly so that its distal end is introduced into a target vascular site;
(b) passing a vascular embolization device comprising an expansible embolizing element through the microcatheter so that it emerges from the distal end of the microcatheter into the target vascular site; and
(c) applying a magnetic field to hold the device in the vascular site while permitting the embolizing element to expand in situ substantially to fill remaining volume of the target vascular site while retaining the embolizing element on the carrier.

40. A method for embolizing a target vascular site having a defined volume, comprising the steps of:
(a) passing a microcatheter intravascularly so that its distal end is introduced into a target vascular site;
(b) passing a vascular embolization device comprising an expansible embolizing element through the microcatheter so that it emerges from the distal end of the microcatheter into the target vascular site; and
(c) applying a magnetic field to hold the device in the vascular site while permitting the embolizing element to expand in situ substantially to fill remaining volume of the target vascular site while retaining the embolizing element on the carrier.

41. A method for embolizing a target vascular site having a defined volume, comprising the steps of:
(a) passing a microcatheter intravascularly so that its distal end is introduced into a target vascular site;
(b) passing a vascular embolization device comprising an expansible embolizing element through the microcatheter so that it emerges from the distal end of the microcatheter into the target vascular site; and
(c) applying a magnetic field to orient the device in the vascular site while permitting the embolizing element to expand in situ substantially to fill remaining volume of the target vascular site while retaining the embolizing element on the carrier.

42. A device for embolizing a vascular site, comprising:
an elongate, filamentous carrier formed of a flexible material having an elastic memory;
at least one expansible embolizing element non-releasably carried on the carrier at spaced intervals along the length of the carrier;
at least one magnetically responsive element associated with the carrier or embolizing element.

43. The device according to claim 42 wherein the at least one magnetically responsive element comprises a plurality of particles disbursed in the embolizing element.

44. The device according to claim 42 wherein the at least one magnetically responsive element comprises a body attached to the filament.

45. The device according to claim 42 wherein the at least one magnetically responsive element comprises the magnetically responsive material comprising the filament.

46. A device for embolizing a vascular site, comprising:
an elongate, filamentous carrier formed of a flexible material having an elastic memory;
a plurality of expansible embolizing elements located at spaced intervals along the length of the carrier;
and at least one magnetically responsive element associated with the filament or the embolizing element.

47. A vascular implant device for embolizing a vascular site, wherein the device has an initial configuration in which it is in the form of a model of the vascular site, and wherein the device is compressible from the initial configuration into a compressed configuration, and expansible from the compressed configuration into an expanded configuration substantially conforming to the shape and size of the vascular site, and a magnetically responsive element therein capable of aligning the device in an applied magnetic field of at least 0.05T.

48. The vascular implant device of claim 47, wherein the device is formed of a hydrophilic foam material.

49. The vascular implant device of claim 48, wherein the foam material is a macroporous hydrogel foam material.

50. The vascular implant device of claim 47, wherein the device is radiopaque.

51. The vascular implant device of claim 47, wherein the initial configuration of the device is in the form of a scaled-down model of the vascular site.

52. A vascular implant device for embolizing a vascular site, the device having a compressed configuration from which it is expansible into an expanded configuration substantially conforming to the shape and size of the vascular site, wherein the device is substantially formed of a hydrophilic foam material, and a magnetically responsive element for aligning the device in an applied magnetic field of at least about 0.05T.

53. The vascular implant device of claim 52, wherein the implant device has an initial configuration in which it is in the form of a model of the vascular site, and wherein the device is compressible from the initial configuration into a compressed configuration, and expansible from the compressed configuration into an expanded configuration substantially conforming to the shape and size of the vascular site.

54. The vascular implant device of claim 53, wherein the initial configuration of the device is in the form of a scaled-down model of the vascular site, from which it is compressible into the compressed configuration.

55. An embolic material for occluding vascular defects, the embolic material comprising a plurality of magnetically responsive particles each coated with a hydrogel that expands when contacted with blood.

56. The embolic material according to claim 55 wherein the particles have a longest dimension of less than about 50 nm.

57. The embolic material according to claim 55 wherein the particles have an average longest dimension of between about 5 nm and about 50 nm.

58. The embolic material according to claim 55 wherein the hydrogel on the particles expands to a thickness of at least about 5 nm.

59. The embolic material according to claim 55 wherein the hydrogel on the particles expands on contact with blood to a thickness of at least 1.5 times the longest dimension of the particle.

60. The embolic material according to claim 55 wherein the hydrogel on the particles has a thickness of between about 2.5 nm and about 20 nm prior to contact with blood, and a thickness of between about 5 nm and about 50 nm after contact with blood.

61. The embolic material according to claim 55 wherein material has a magnetic density of between about 0.5 g/cc and about 2 g/cc before the hydrogel expands, and wherein the material has a magnetic density of between about 0.75 g/cc and about 3 g/cc after the hydrogel expands.

62. The embolic material according to claim 55 wherein material has a magnetic density of less than about 0.5 g/cc after the hydrogel expands.

63. The embolic material according to claim 55 wherein the force on the embolic material prior to expansion of the hydrogel in a magnetic gradient of 0.5 T is at least 0.5 g/cc, and the magnetic force on the embolic material after expansion of the hydrogel in a magnetic gradient of 1 T/m is less than 5 g/cc.

64. The embolic material according to claim 55 wherein the force on the embolic material prior to expansion of the hydrogel in a magnetic gradient of 0.5 T is at least 0.5 g/cc, and the magnetic force on the embolic material after expansion of the hydrogel in a magnetic gradient of 10 T/m is less than 5 g/cc.

65. An embolic material comprising an expansible hydrogel body with a magnetically responsive material associated therewith, the magnetic material creating a pulling force of at least about 0.5 g/cc in a magnetic gradient of 0.5T/m, prior to expansion of the hydrogel.

66. The embolic material according to claim 65 wherein the magnetic material comprises an element of a magnetically responsive material embedded in the hydrogel body.

67. The embolic material according to claim 65 wherein the magnetic material comprises a magnetically responsive coil.

68. The embolic material according to claim 65 wherein the magnetically responsive material comprise particles of magnetic material embedded in hydrogel.

69. The embolic material according to claim 68 wherein the magnetic density of the magnetic material before expansion of the hydrogel is greater than about 0.75 percent by volume and wherein the density of the magnetic material after expansion of the hydrogel is less than about 0.5 percent by volume.

70. The embolic material according to claim 65 wherein the pulling force on the body in a gradient of 0.5 T/m is at least about 0.5 g/cc prior to expansion of the hydrogel, and the pulling force on the body in a gradient of 0.5 T/m is less than about 0.25g/cc after expansion of the hydrogel.

71. The embolic material according to claim 65 wherein the magnetic material is a magnetic element alignable with an applied magnetic field of at least 0.05T to orient the body in a selected orientation.

72. A method of embolizing a vascular defect, the method comprising introducing an embolic material comprising an expansible hydrogel having a magnetic material associated therewith adjacent the vascular defect; and applying a magnetic gradient to the vascular defect to draw the embolic material into the vascular defect.

73. A method of embolizing a vascular defect, the method comprising introducing an embolic material comprising an expansible hydrogen having a magnetically responsive material associated therewith into the vascular defect, and applying a magnetic gradient to the vascular defect to hold the embolic material in the vascular defect.

74. A method of embolizing a vascular defect, the method comprising introducing an embolic material comprising an expansible hydrogel having magnetically responsive material associated therewith, an applied magnetic gradient of at least 0.5 T/m creating a pulling force of at least 0.5 gm/cc on the embolic prior to expansion of the hydrogel.

75. The method according to claim 74 wherein after expansion of the hydrogel, an applied magnetic gradient of at least 1 T/m creating a pulling force of no more than 1 gm/cc.

76. The method according to claim 74 wherein after expansion of the hydrogel, an applied magnetic gradient of at least 10 T/m creating a pulling force of no more than 1 gm/cc.

77. The method of embolizing a vascular defect comprising introducing an embolic material comprising an expansible hydrogel having magnetically responsive material associated therewith into the vascular defect, and applying a magnetic field to the vascular defect in a direction to orient the hydrogel body in a desired orientation within the vascular defect.

78. The method of embolizing a vascular defect comprising introducing an embolic material comprising an expansible hydrogel having magnetically responsive material associated therewith into the vascular defect, and applying a magnetic field to the vascular defect in a direction to orient the hydrogel body in a desired orientation within the vascular defect, and applying a magnetic gradient to the vascular defect to retain the embolic in the defect.
